(19) United States
(12) Patent Application Publication
(10) Pub. No.: US 2022/0378608 A1
(43) Pub. Date: Dec. 1, 2022

(54) DEVICES, SYSTEMS, AND METHODS FOR MODULATING TISSUE TEMPERATURE

(71) Applicant: DUKE UNIVERSITY, Durham, NC (US)

(72) Inventors: Syed Faaiz ENAM, Durham, NC (US); Mark CALHOUN, Durham, NC (US); Tarun SAXENA, Durham, NC (US); Steven Owen, Durham, NC (US); Ravi BELLAMKONDA, Durham, NC (US); Reed CHEN, Durham, NC (US); Paolo MACCARINI, Durham, NC (US)

(21) Appl. No.: 17/769,985

(22) PCT Filed: Oct. 16, 2020
(86) PCT No.: PCT/US2020/056078

§ 371 Date: Apr. 18, 2022

Related U.S. Application Data
(60) Provisional application No. 62/923,081, filed on Oct. 18, 2019.

(51) Int. Cl. A61F 7/12

(52) U.S. Cl. CPC A61F 7/12; A61F 2007/0002

(57) ABSTRACT

This present disclosure provides devices, systems, and methods relating to modulating the temperature of a target tissue in a subject for various therapeutic purposes. In particular, the present disclosure provides devices, systems, and methods for the focal delivery of cytostatic hypothermia to a target tissue to treat a disease or condition in a subject (e.g., cancerous tumors).

### RELATED APPLICATIONS

This application claims priority to and the benefit of U.S. Provisional Patent Application No. 62/923,081 filed Oct. 18, 2019, which is incorporated herein by reference in its entirety for all purposes.

### FIELD

This present disclosure provides devices, systems, and methods relating to modulating the temperature of a target tissue in a subject for various therapeutic purposes. In particular, the present disclosure provides devices, systems, and methods for the focal delivery of cystostatic hypothermia to a target tissue to treat a disease or condition in a subject (e.g., cancerous tumors).

### BACKGROUND

Patients with malignant brain tumors such as glioblastoma multiforme (GBM) have a median survival of 15-18 months and only 7% survive 5 years after diagnosis and treatment. This is in part because of the limitations of standard-of-care treatment (surgery, chemotherapy, and radiotherapy). For example, surgical resection can only remove visible and bulk mass and cannot reach any remaining cells that may have infiltrated deeper. These remaining cells are the source of GBM recurrence which happens in nearly all patients with GBM. Chemotherapies are hindered by the blood-brain-barrier, neurotoxicity, the evolution of resistance. Radiotherapy lacks tumor specificity and leads to indiscriminate damage tumor and healthy brain alike. To circumvent these limitations, we provide here a device and method using focal, cytostatic hypothermia to stunt the growth of tumors and its use as an adjuvant to facilitate tumor killing.

### SUMMARY

Embodiments of the present disclosure include a thermoelectric cooling device for reducing temperature of a target tissue in a subject. In accordance with these embodiments, the device includes a thermoelectric modulator, a heat exchange system functionally coupled to the thermoelectric modulator, and at least one thermally conductive probe functionally coupled to the thermoelectric modulator. In some embodiments, activation of the thermoelectric modulator transfers heat from a target tissue to the heat exchange system, thereby reducing the temperature of the target tissue.

In some embodiments, the thermoelectric modulator comprises a Peltier module. In some embodiments, the Peltier module comprises a Peltier cell coupled to the at least one thermally conductive probe. In some embodiments, the thermoelectric modulator comprises a plurality of Peltier modules, wherein each Peltier module comprises a Peltier cell coupled to the at least one thermally conductive probe. In some embodiments, the Peltier module comprises a thermally conductive base plate positioned between the Peltier cell and the at least one thermally conductive probe.

In some embodiments, the thermoelectric modulator comprises applying electrical power to the thermoelectric modulator to cause heat to be transferred from the target tissue to the heat exchange system.

In some embodiments, the heat exchange system comprises a fan to facilitate the transfer of heat from the target tissue. In some embodiments, the heat exchange system comprises a fluid block to facilitate the transfer of heat from the target tissue. In some embodiments, the fluid block comprises a piping system and a fluid. In some embodiments, the piping system is coupled to the thermoelectric modulator and the fluid flows through the piping system, thereby facilitating the transfer of heat from the target tissue.

In some embodiments, the fluid comprises a biocompatible coolant.

In some embodiments, the thermoelectric modulator comprises a power source.

In some embodiments, the heat exchange system comprises a magnetic motor. In some embodiments, the heat exchange system comprises a non-magnetic motor.

In some embodiments, the at least one thermally conductive probe is in direct contact with the target tissue. In some embodiments, the at least one thermally conductive probe is comprised of a biocompatible material.

In some embodiments, the device further comprises a target tissue temperature monitoring device configured to be in direct contact with the target tissue.

In some embodiments, the device further comprises a control system. In some embodiments, the control system is configured to modulate the temperature of the target tissue by adjusting one or more device parameters to increase or decrease heat transfer from the target tissue.

In some embodiments, the temperature of the target tissue is reduced to about 20° C. to about 35° C.

In some embodiments, the target tissue comprises a tumor. In some embodiments, the target tissue comprises a glioblastoma.

Embodiments of the present disclosure also include a method of treating a target tissue in a subject. In accordance with these embodiments, the method includes activating any of the devices described above. In some embodiments, activation of the device transfers heat from the target tissue to the heat exchange system, thereby reducing the temperature of the target tissue.

Embodiments of the present disclosure also include a method of treating a tumor in a subject. In accordance with these embodiments, the method includes implanting a thermoelectric cooling device in a subject, the device comprising a thermoelectric modulator, a heat exchange system functionally coupled to the thermoelectric modulator, and at least one thermally conductive probe functionally coupled to the thermoelectric modulator and in direct contact with the tumor. In some embodiments, the method includes activating the thermoelectric modulator such that it facilitates the transfer of heat from the tumor to the heat exchange system, thereby inducing cytostatic hypothermia in the tumor.

In some embodiments, the method includes inducing cytostatic hypothermia in the tumor by reducing the temperature of the tumor to about 20° C. to about 35° C.

In some embodiments, treating the tumor includes cycling between activating the thermoelectric modulator for a period of time and deactivating the thermoelectric modulator for a period of time.

In some embodiments, treating the tumor includes adjusting one or more device parameters to modulate heat transfer from the target tissue, thereby increasing or decreasing the temperature of the target tissue. In some embodiments, modulating heat transfer decreases the temperature of the tumor as compared to a base temperature. In some embodiments, modulating heat transfer increases the temperature of the tumor as compared to a base temperature.

In some embodiments, the method further includes subjecting the subject to a magnetic resonance imaging (MRI) procedure.

In some embodiments, the subject is receiving chemotherapy. In some embodiments, the chemotherapy includes administration of temozolomide.

In some embodiments, the subject is receiving cancer immunotherapy. In some embodiments, the cancer immunotherapy comprises administration of CAR T cells, checkpoint inhibitors, or antibody-based treatment.

### BRIEF DESCRIPTION OF THE DRAWINGS

FIG. 1 includes a representative cross-sectional view of a thermoelectric cooling device, according to one embodiment of the present disclosure.

FIG. 2 includes a representative diagram of a thermoelectric cooling device and system for the treatment of a brain tumor, according to one embodiment of the present disclosure.

FIGS. 3A-3E include a representative images of a thermoelectric cooling device that is MRI-compatible for use in subjects that require regular MRI imaging as part of therapeutic monitoring (FIGS. 3A-3D). FIG. 3E is a representative cutaway diagram of the corresponding implantable portion of the device.

FIGS. 4A-4D include representative images of a thermoelectric cooling device that includes a fluid block and piping system (FIG. 4A, perspective view; FIG. 4B, cross-sectional view) to facilitate the transfer of heat from the target tissue via fluid flow through the fluid block (FIG. 4D), according to one embodiment of the present disclosure. This embodiment of the thermoelectric cooling device can be fully implanted into a subject (FIG. 4C).

FIGS. 5A-5C representative images of a thermoelectric cooling device that includes a fluid block and piping system (FIGS. 5A-5B), which demonstrated that the addition of fluid flow/circulation through the system significantly enhanced temperature regulation (e.g., cumulative 30 ml/s flow (10 ml/s per pipe) reducing the scalp temperature to 38° C. and 39.5° C. at 10 W and 20 W, respectively).

FIGS. 6A-6D include computational simulations of one or more thermally conductive probes (FIGS. 6A-B) and representative images of a thermoelectric cooling device that includes a multi-probe array design (FIG. 6C) and a Peltier grid (FIG. 6D). The multiprobe design demonstrated more homogenous temperature distribution (FIG. 6B) as compared to a single probe configuration (FIG. 6A) and the ability to remain in a relevant cytostatic hypothermia range.

FIGS. 7A-7C: Effect of continuous and intermittent cytostatic hypothermia on in vitro tumor cell division. (FIG. 7A) Tumor cell growth curves quantified by area coverage of the well at 37° C., 30° C., and 25° C. Plates were left to grow overnight at 37° C. after seeding and switched to their respective incubator the following morning after imaging (Day 0); (FIG. 7B) Tumor cell growth curves at 37° C., 25° C., and 20° C. These were left to grow in 37° C. until they reached ˜20% confluence and then transferred to their respective incubator (Day 0); (FIG. 7C) Intermittent hypothermia assessment on tumor growth across the well. Cells that received hypothermia were plated and grown for two days at 37° C., followed by 3 days under 25° C. hypothermia, and then were transferred between the two incubators. H00=0 hours of hypothermia/day, H08=8 h/day hypothermia, H16=16 h/day hypothermia, HH16=16 h/day of 20° C. hypothermia, H24=24 h/day of hypothermia.

FIGS. 8A-8C: Mechanism and effects of hypothermia on tumor cell lines. (FIG. 8A) Percentage of cells in each stage of the cell cycle after either 3 days of 37° C., 6 days of 25° C., or 10 days of 25° C. Cells that showed polyploidy are not shown in these graphs. (FIG. 8B) Amount of intracellular ATP from the tumor cell lines when grown for 3 days at 37° C., 6 days at 25° C., or 10 days at 25° C. (FIG. 8C) Concentration of cytokines (IL-6 and IL-8) collected in the media at different time points and temperatures.

FIG. 9: Effect of cycling temperature. Circles represent tumor kept at 37° C. for 24 hours every day (e.g., 25° C. for 0 hours every day) and squares represent tumors kept at 25° C. for 24 hours every day. Triangles represent tumors kept at 25° C. for 24 hours a day until day five. After this, the cells were kept at 25° C. for 16 hours and 37° C. for 8 hours every day. These data indicate that 25° C. halts some cells in the middle of mitoses, which is completed during the rewarming phase (day 6). As shown, the cycled tumor cells lost well coverage faster than the continuously cooled tumors.

FIGS. 10A-10B: Use of cytostatic hypothermia with chemotherapy and CAR T immunotherapy. (FIG. 10A) Growth curves of cells with concomitant TMZ chemotherapy and hypothermia. Gray line represents treatment with TMZ at 37° C. while blue line represents treatment with TMZ at 25° C. Plotted lines show growth curves after the final wash of media to remove TMZ, both at 37° C. (FIG. 10B) GFP+EGFR+CT2A tumor cell counts with treatment of CAR T cells and continuous or intermittent hypothermia. Control represents no addition of CAR T cells. H00=0 h/day hypothermia, H20=20 h/day hypothermia, H24=24 h/day hypothermia.

FIGS. 11A-11H: Focal in vivo delivery of cytostatic hypothermia. (FIG. 11A) Model of Peltier-powered device. Implantable portion consists of gold needle, copper plate, and polycarbonate base with a thermistor. Removable portion consists of polycarbonate plate with a Peltier, aluminum heat sink, and fan. (FIG. 11B) Finite-element model of rat brain with a 1-mm probe cooled to 0° C., to cool a spherical tumor such that the periphery reaches 25° C. Temperatures return to 37° C. 5 mm from the probe. (FIG. 11C) In vivo temperature measurement from the thermistor in the implant, 1-mm from the probe. Here, both rats were implanted with the device, but only the hypothermia (blue) one was switched on. (FIG. 11D) Representative MRI images of tumor growth on Day 0 (after switching device on) and Day 7. Upper row is with hypothermia, bottom row is control. (FIG. 11E) Tumor volume measured from MRI and normalized to Day 0 volume. (FIG. 11F) Rat weight measured across the survival study. Red bars indicate rats with devices switched off, while blue lines indicate rats receiving focal hypothermia. (FIG. 11G) Kaplan-Meier plot depicting survival time of rats with the device switched off (red) or switched on to deliver hypothermia (blue). (FIG. 11H) Cross section of brain slice from earliest deceased rat indicating an external tumor that pressed on the brain and was not near the cooling probe. The internal tumor is also visible with greater nuclear staining and debris.

FIGS. 12A-12B: Focal in vivo delivery of cytostatic hypothermia. Kaplan-Meier plot depicting survival time of rats with the device switched off (red) or switched on to deliver hypothermia (blue) in Fischer rats with F98 GBM (FIG. 12A) and RNU rats with human U87-MG GBM (FIG. 12B).

### DETAILED DESCRIPTION

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

“Therapy” and/or “therapy regimen” generally refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, the treatment comprises the treatment, alleviation, and/or lessening of pain.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Tissue Modulation Devices

Even after standard-of-care treatment (e.g., surgery, chemotherapy, and radiation), patients with glioblastoma (GBM) have a median survival of 15-18 months. Furthermore, only 7% of patients survive 5 years after their diagnosis. This is likely due to the ineffectiveness of current therapies in eradicating GBM. This results in GBM recurrence, of which >80% of recurrent GBMs are local (3-5) and only 20-30% can be resected again before recurring once more. Unfortunately, patients have few alternative therapeutic options. Chemotherapies are hindered by the blood-brain-barrier and the evolution of resistance. Similarly, monoclonal antibodies that reduce angiogenesis may mildly improve symptoms but have little effect on survival.

This dismal performance has necessitated exploring alternate strategies outside the realm of pharmaceuticals and into that of physical phenomena. The first, albeit mild, success was through enabling sustained release of a chemotherapeutic (via an intratumorally implanted Gliadel wafer). The second used electroceutical “tumor-treating field therapies” (TTFTs) which may have had a mild effect on overall survival but is controversial. Recently, a novel device composed of aligned nanofibers to promote directional GBM migration received FDA breakthrough status, underscoring the need for new approached to this disease.

There are also alternative strategies, one of which involves adaptive therapy. With this strategy, the goal of therapy becomes to maintain a tumor burden by adjusting therapeutic dose. This is because when chemotherapy is administered, only chemosensitive cells perish. This provides an empty and nutrient rich field for more aggressive chemoresistant cells to proliferate. By controlling the dose of chemotherapy, the load of chemosensitive can be ‘maintained’ so that they keep a check on chemoresistant cells. This strategy has prolonged survival in animal models. Merging the idea of maintaining a tumor burden with the use of a physical force, a focal hypothermia approach was developed.

Hypothermia has neuroprotective properties after brain injuries and can reduce tumor division. It reduces metabolism, oxygen and glucose consumption, and reduces edema, excitotoxicity, and free-radical formation. However, whole-body hypothermia can weaken the body's immune system and enables tumor proliferation. Instead, there is significant promise in focal hypothermia. Cortical cooling devices successfully halt seizures in primates and intraoperatively in patients. For tumor therapies, focal hypothermia has been used to ablate tumor mass via subzero temperatures and cryosurgery. In the 1950s, cryoprobes tethered to large refrigeration machines were transiently implanted to ablate intracranial tumors. While these were safe, they were ultimately unsuccessful.

Instead of ablation, the present disclosure explored the use focal hypothermia to identify a window of “cytostatic hypothermia” wherein tumor division is halted but healthy brain is left unharmed. As described further herein, the depth and duration of hypothermia on multiple human GBM lines in vitro was tested, which was followed by an examination of the effects of hypothermia on chemotherapy and CAR T immunotherapy in vitro, ultimately leading to the development of devices, systems, and methods to deliver hypothermia in vivo.

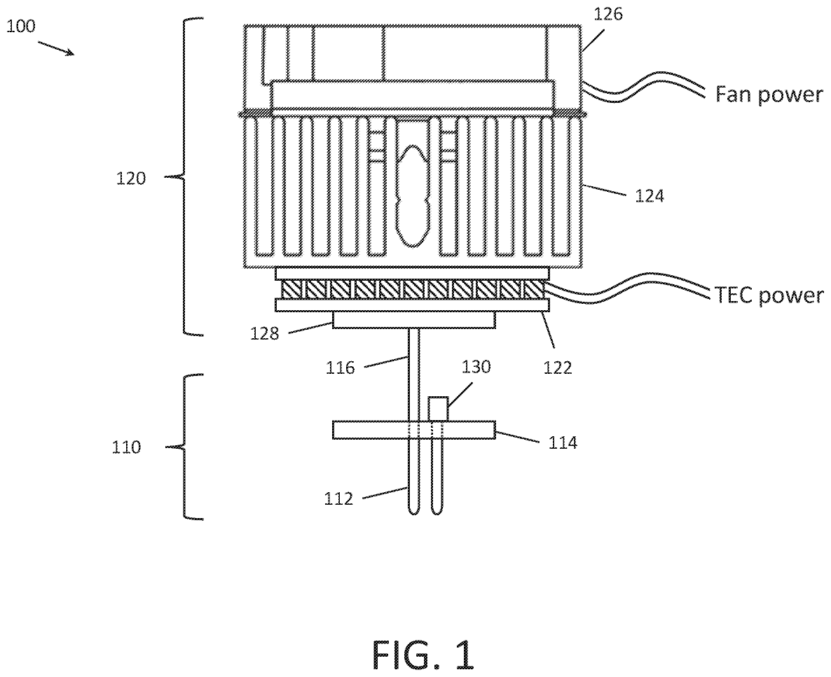

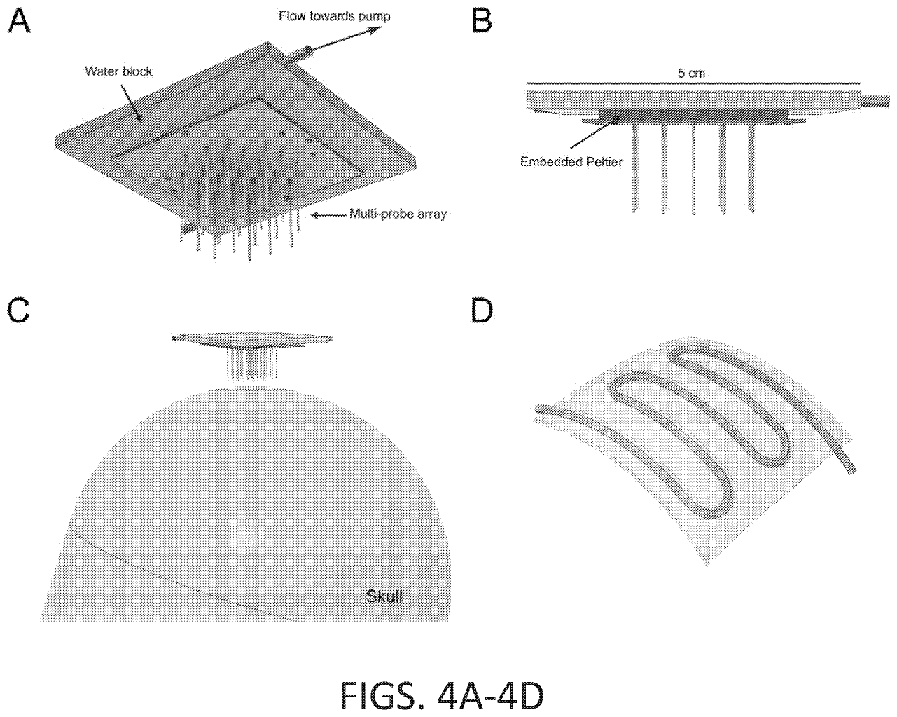

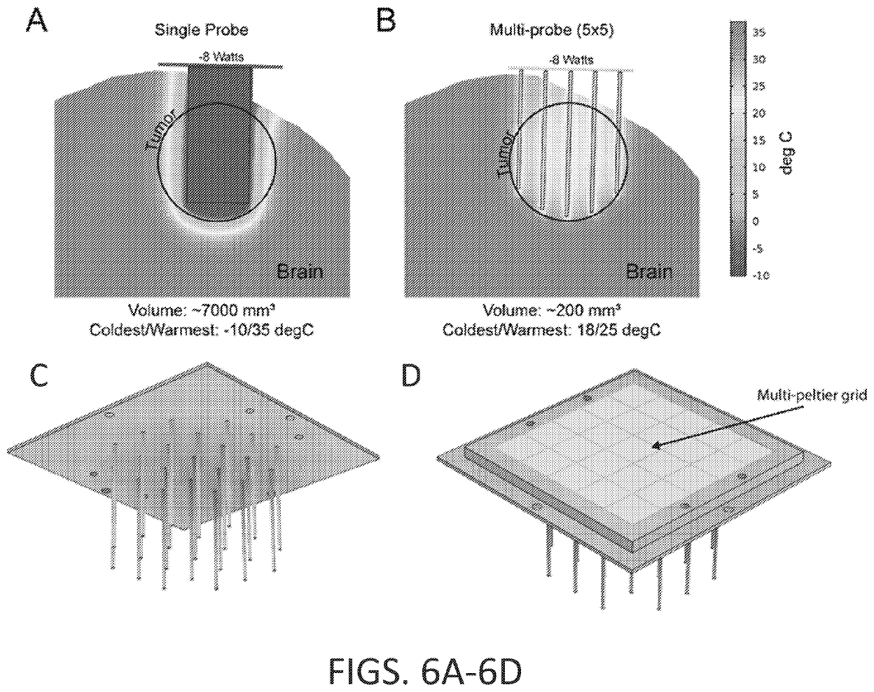

Embodiments of the present disclosure provides devices, systems, and methods relating to modulating the temperature of a target tissue in a subject for various therapeutic purposes. In particular, the present disclosure provides devices, systems, and methods for the focal delivery of cystostatic hypothermia to a target tissue to treat a disease or condition in a subject (e.g., cancerous tumors). As shown in FIG. 1, embodiments of the present disclosure include a thermoelectric cooling device (100) for reducing temperature of a target tissue in a subject. The thermoelectric cooling device (100) can include an implantable portion (110) and a heat exchange system (120). Generally, the implantable portion (110) includes at least one thermally conductive probe (112) functionally coupled to a thermoelectric modulator (122), and an attachment base (114). In some embodiments, the thermally conductive probe (112) is adjustably attached to the attachment base (114), which is used to position the thermally conductive probe (112) at the site of a target tissue (e.g., in direct contact with a target tissue). In some embodiments, the thermally conductive probe (112) is formed from a substantially rigid, non-ferrous, and thermally conductive material, which is also bio-compatible (e.g., gold-plated or graphite-based). In some embodiments, the thermally conductive probe (112) comprises cytotoxic elements (e.g., coated with a cytotoxic agent). In one embodiment, the thermally conductive probe (112) can be in the form of a probes or needle. In another embodiment, thermally conductive probe (112) can include a heat pipe. This allows precise placement of the probe in the tumor to be treated (e.g., the treatment site) and is safe for use in or near a magnetic resonance imaging (MRI) machine (e.g., comprised of non-ferrous material). The geometry of the thermally conductive probe (112), including the length, diameter, shape, and the like can be adjusted depending on the size and location of the target tissue and the particular characteristics of the subject being treated. For example, the length and width of the thermally conductive probe (112) (e.g., needle) and/or the angle of protrusion can be modified. The number of probes used can also be increased without departing from the scope of the disclosure (see, e.g., multiprobe configurations in FIGS. 4 and 6). In some embodiments, the thermally conductive probe (112) includes insulated portions, with some sections that are thermally conductive and other sections that are not, so as to protect tissue that is not meant to be cooled. In some embodiments, the thermally conductive probe (112) have various geometries to ensure homogenous tissue cooling (e.g., cylindrical, horizontal, round, mesh-like, branching pattern).

In some embodiments, the implantable portion (110) is connected in a thermally conductive manner to the heat exchange system (120), optionally using a thermal connector element (116). In some embodiments, the thermal transfer element is a wire formed of a heavy-gauge solid copper wire. In other embodiments, wire can be a rigid rod-like element formed of a material with low thermal resistance, or the wire can be multiple types of thermally conductive material coupled together. Wire (116) can also include a connector for detaching the heat exchange system (120) from implantable portion (110). In other embodiments, the thermal connector element (116) is not limited to a wire, but can use other types of conductive, convective, or radiative connection. In some embodiments, the probe can also have insulating sleeves/sections so that cooling can be limited in normal brain tissue or parts of the tumor that have smaller diameter.

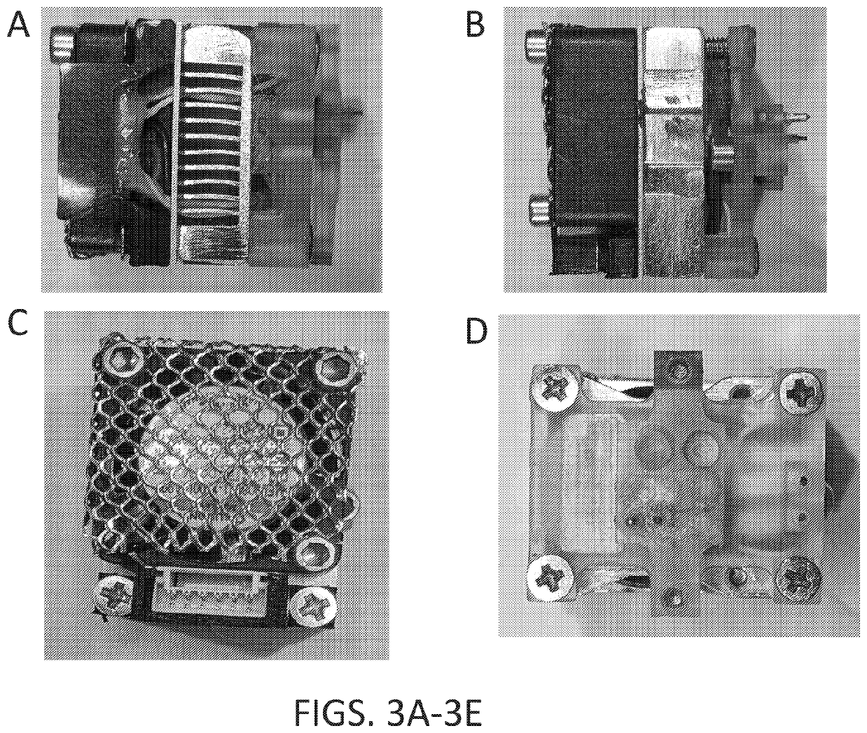

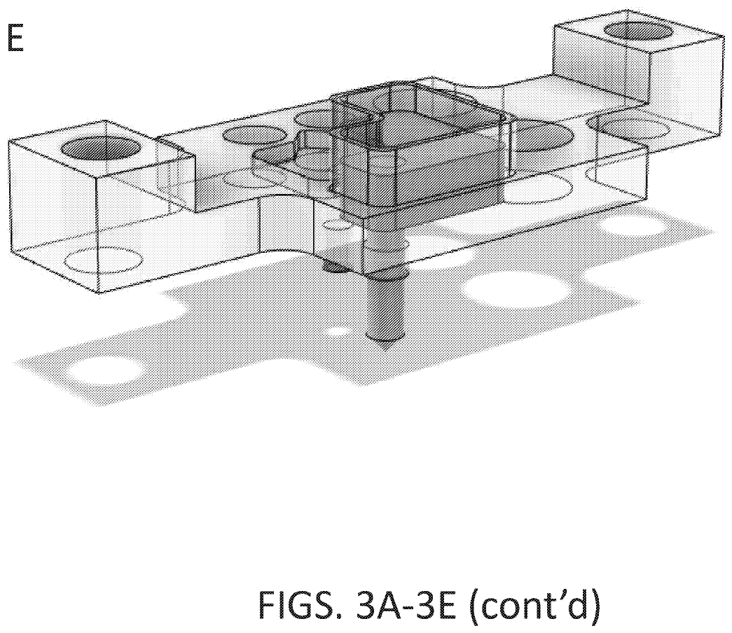

In some embodiments, the heat exchange system (120) includes at least one thermoelectric modulator (122). In some embodiments, the heat exchange system (120) is configured to be separate from the thermoelectric modulator (122). In some embodiments, the heat exchange system comprises a magnetic motor. In other embodiments, the heat exchange system comprises a non-magnetic motor (e.g., for MRI compatibility). In some embodiments, the thermoelectric modulator (122) is MRI-compatible in that it lacks magnetic and/or metallic components that would be considered by one of ordinary skill in the art to be problematic for use in an MRI machine. The thermoelectric modulator (122) is generally configured to transfer heat from a target tissue to the heat exchange system (120), thereby reducing the temperature of the target tissue. The thermoelectric modulator (122) can be any suitable device for modulating temperature (increasing and/or decreasing), such as a cold plate, heat exchanger, heat pump, thermoelectric cooler (TEC), etc., or a combination thereof. The thermoelectric modulator (122) can also employ any suitable mechanism for transferring heat, such as liquid cooling, convection cooling, and the like (see, e.g., FIGS. 3-6). In one embodiment, the thermoelectric modulator (122) is in the form of a TEC (thermoelectric cooler), also known as a Peltier device. For example, when a current is applied to the Peltier device (also referred to as a Peltier module), heat is transferred from one side of the device to the opposite side, creating a “hot” side and a “cold” side. As the applied current increases, the temperature difference between the two halves also increases, and thus the device can be “tuned” to a prescribed ΔT.

In some embodiments, to increase the range of the heat differential, a heat sink (124) can be attached to the hot side of the thermoelectric modulator (122), which maintains the temperature of the hot side at a level within an operating range of the TEC. Heat sink (124) can also be convection-cooled with an integrated fan (126), and/or as shown in FIGS. 4-6, the heat sink can be a fluid block comprising a piping system and a fluid (e.g., water block). The heat sink can also be a vapor chamber (e.g., heat spreader). The fluid used in these embodiments can be any biocompatible coolant (e.g., water, ethanol), including any aqueous fluid capable of flowing through the piping system to transfer heat from the target tissue. The fluid block could be made of various materials, including but not limited to, gold-plated copper. In some embodiments, the fluid block is made of 3D printed metal to enable easier construction of curvature relative to the skull of a subject. In some embodiments, the fluid block is detachably coupled to a motor (e.g., piezoelectric or electromagnetic motor) that drives fluid flow through the piping system. In some embodiment, the flow rate of the pump is at least about 1 ml/s to 50 ml/s. In some embodiment, the flow rate of the pump is at least about 1 ml/s to 40 ml/s. In some embodiment, the flow rate of the pump is at least about 1 ml/s to 30 ml/s. In some embodiment, the flow rate of the pump is at least about 1 ml/s to 20 ml/s. In some embodiment, the flow rate of the pump is at least about 1 ml/s to 10 ml/s. In some embodiment, the flow rate of the pump is at least about 5 ml/s to 50 ml/s. In some embodiment, the flow rate of the pump is at least about 10 ml/s to 50 ml/s. In some embodiment, the flow rate of the pump is at least about 20 ml/s to 50 ml/s. In some embodiment, the flow rate of the pump is at least about 40 ml/s to 50 ml/s. In some embodiment, the flow rate of the pump is at least about 5 ml/s to 10 ml/s. In some embodiment, the flow rate of the pump is at least about 6 ml/s to 11 ml/s. In some embodiment, the flow rate of the pump is at least about 7 ml/s to 12 ml/s. In some embodiment, the flow rate of the pump is at least about 8 ml/s to 13 ml/s. In some embodiment, the flow rate of the pump is at least about 9 ml/s to 14 ml/s. In some embodiment, the flow rate of the pump is at least about 10 ml/s to 15 ml/s.

The heat sink (124) can be designed for cooling by any means, including but not limited to natural convection, liquid cooling, a remotely located fan, or any other type of cooling. The heat exchange system (120) can also include a plate (128) on the cold side of the thermoelectric modulator (122), including, for example, positioned between one or more Peltier modules and at least one thermally conductive probe. Base plate (128) can be, for example, a copper plate, which can optionally be integrated into a non-conductive assembly structure (see, e.g., FIG. 3). Base plate (128) can connect to the wire (116) and conduct the thermally conductive probe (112).

In some embodiments, the thermoelectric cooling device (100) includes at least one Peltier module (see, e.g., FIG. 6) as part of the thermoelectric modulator (122). In some embodiments, the Peltier module comprises a Peltier cell coupled to the at least one thermally conductive probe (112). In some embodiments, the thermoelectric modulator (122) comprises a plurality of Peltier modules, and each Peltier module comprises a Peltier cell coupled to the at least one thermally conductive probe (122). In some embodiments, each Peltier cell includes insulative material such that each cell is separately sealed from other Peltier cells to limit cross-heating or cross-cooling. In some embodiments, the Peltier cell and/or the Peltier module is MRI-compatible in that it lacks magnetic and/or metallic components that would be considered by one of ordinary skill in the art to be problematic for use in an MRI machine.

In some embodiments, the Peltier module comprises a thermally conductive base plate (128) positioned between the Peltier cell and the at least one thermally conductive probe (122). In some embodiments, the heat exchange system (120) is provided in a portable form that is fully implantable within a subject (see, e.g., FIG. 4), such that the subject can be at least somewhat mobile while undergoing treatment. In some embodiments, the thermoelectric cooling device includes a fluid-based cooling system where cold fluid passes through the probe. The cold fluid can be maintained at a low somewhere else in the body through a Peltier module, for example. In some embodiments, the Peltier module is hermetically sealed and implanted in or on the skull of the subject being treated. In other embodiments, one or more components of a fluid-based cooling system (e.g., fluid block or fluid pump) can be positioned in or on a subject's body at a location that is separate from the other components of the thermoelectric cooling device (e.g., the heat exchange system). In still other embodiments, one or more heat sinks can be positioned in or on a subject's body at a location that is separate from the other components of the thermoelectric cooling device (e.g., the heat exchange system).

In some embodiments, a desired target tissue temperature can be achieved either by providing one or more target tissue temperature monitoring devices and a controller. In the example embodiment of FIG. 1, a temperature monitoring device (130) is placed adjacent to the thermally conductive probe (112). The temperature monitoring device (130) can be a conventional temperature monitoring device, such as a thermocouple or thermistor. The temperature monitoring device (130) can be operated independently from the thermoelectric cooling device (100), or it can be integrated into a control system functionally coupled to the thermoelectric cooling device (100) for controlling the source temperature of the cooling device. In some embodiments, the control system is configured to modulate the transfer of heat to or from the target tissue by adjusting or more device parameters to increase or decrease from the temperature of the target tissue. In some embodiments, the temperature monitoring device (130) can be located on the thermoelectric modulator (122) for recording the temperature of the cold side of cooling device, or a temperature monitoring device can be included on the hot side of the device to limit adverse effects to the subject's skin, for example. This can advantageously be used in a closed-loop control system to maintain a desired temperature at the treatment site. In another embodiment, the temperature monitoring device (130) can be detachably coupled to implantable portion (110) such that it is removable (e.g., when the patient undergoes MRI).

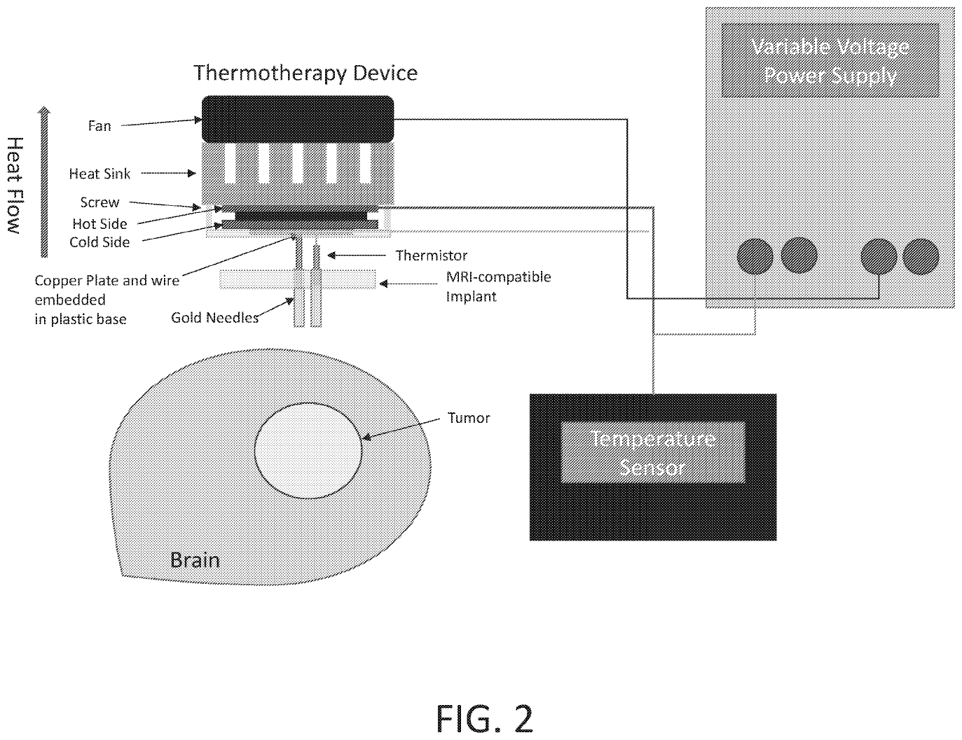

In some embodiments, the thermoelectric cooling devices (100) are integrated into a system for delivering focal hypothermia to a desired target tissue (e.g., brain tumor). As shown in FIG. 2, the system can include the device (100) and additional components that supply power to the device (e.g., to the thermoelectric modulator, the fan, the fluid block, etc.), and/or integrate with the temperature sensor. In some embodiments, the thermoelectric modulator is connected to a power source that is external to a subject with a transcutaneous wire/lead. In other embodiments, the thermoelectric modulator includes a power source that supplies power wirelessly and is implanted in the body of a subject away from the skull. In some embodiments, the system includes a controller or control system that allows a user to adapt the therapy to a particular subject and to a particular disease or condition. In some embodiments, the thermoelectric cooling devices and systems of the present disclosure can be used to treat a brain tumor in a subject. For example, the implantable portion (110) can be affixed to the skull of the subject using attachment base (114), or the device (100) can be fully implanted in a subject (FIG. 4). The device is generally placed such that at least one thermally conductive probe (112) is in direct contact with the target tissue (e.g., brain tumor). The placement location can be selected such that the low temperature is limited to a relatively small area to slow or halt the growth of the cancerous cells. In some cases, the probe can be provided in the shape of the tumor. Alternately, the probe can pass through the tumor or routed in a path through a core of the tumor. The thermoelectric modulator (122) can then be connected to the heat exchange system (120) and operation of thermoelectric cooling device (100) is initiated. In embodiments in which the thermoelectric modulator is one or more Peltier modules, the device (100) can be connected to power and/or control system, and a current is applied to the modules. The at least one thermally conductive probe (112) is then cooled to a desired temperature below a target temperature of the cold element/tumor.

In some embodiments, the target temperature of a tissue is in the range of approximately 20° C.-35° C. In some embodiments, the target tissue is reduced to about 20° C. to about 30° C. In some embodiments, the target tissue is reduced to about 20° C. to about 25° C. In some embodiments, the target tissue is reduced to about 25° C. to about 35° C. In some embodiments, the target tissue is reduced to about 30° C. to about 35° C. In some embodiments, the target tissue is reduced to about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., or about 35° C.

In particular, it has been demonstrated that the thermoelectric devices and systems disclosed herein can halt growth of multiple human tumor cell lines using temperatures of approximately 25° C.-35° C., and significantly slow down even the most resilient rat tumor cell line, at a target temperature of approximately 20° C.-30° C. This temperature was also shown not to indiscriminately kill cells in the region of the cancerous cells, and therefore has a reduced risk of damage to healthy cells. Further, as most brain cells do not divide, the disclosed range can be a therapeutic window for hypothermia in which GBM growth is halted and yet cells are not killed. In other words, the probe does not act as an ablation mechanism.

In some embodiments, therapy is applied to a target tissue in a consistent and constant manner, with minimal deactivation (e.g., 20-24 hours/day). In other embodiments, cooling is applied to the target tissue in a cyclical manner (e.g., on for a duration, off for a duration). In further embodiments, the cooling element can also include specific temperature cycling, where the cooling source can be actively set to varying temperatures, thus possibly “heating” the tumor. The higher temperature setting can be in the above-mentioned ranges and even include temperatures higher than the body resting temperature, without departing from the definition of the cooling source or departing from the scope of the invention. In other words, the cooling source can also deliver heating to the tumor.

It is to be noted that, although the devices and methods disclosed herein are described with reference to brain tumors, it will be understood by a person of skill in the art that these devices and methods can also be applicable to tumors occurring in other parts of the body.

### 3. Methods of Treatment

Embodiments of the present disclosure include methods of treatment using the thermoelectric cooling devices and systems described herein. Although exemplary methods have been described with respect to the treatment of a brain tumor (e.g., glioblastoma), the devices and systems provided herein can be used to modulate (e.g., increase and/or decrease) the temperature of any target tissue. Additionally, as described further herein, treating a target tissue with the devices and systems of the present disclosure can include administering treatment according to a treatment regimen or protocol established based on various factors, including but not limited to, the type of tissue being treated, specific patient characteristics, the severity of the disease/condition, and the like. In some embodiments, methods of treatment using the thermoelectric cooling devices and systems described herein include treating a solid tumor (e.g., cancers of the lung, breast, prostate, colon, pancrease, rectum, bladder, and the like), and/or treating a liquid tumor (cancers of the blood and bone marrow, such as lymphomas and leukemias).

Accordingly, a particular treatment regimen or protocol can be established for any patient in need thereof, and can include, for example, cycling between decreases and increases in target tissue temperature for a particular period of time, as well as cycling between periods of treatment and non-treatment (on/off cycles any given period of time). Further, as described further herein, treatment can include combination therapies with other treatment modalities. For example, in the context of tumor treatment, a particular treatment regimen or protocol can include combination treatment with an anti-cancer agent (e.g., chemotherapy and/or cancer immunotherapy).

In accordance with the above embodiments, the present disclosure includes methods for treating a tumor in a subject. The method can include implanting a thermoelectric cooling device in a subject. The device can be comprised of a thermoelectric modulator, a heat exchange system functionally coupled to the thermoelectric modulator, and at least one thermally conductive probe functionally coupled to the thermoelectric modulator and in direct contact with the tumor. In some embodiments, the method includes activating the thermoelectric modulator such that it facilitates the transfer of heat from the tumor to the heat exchange system, thereby inducing cytostatic hypothermia in the tumor.

In some embodiments, the method includes inducing cytostatic hypothermia in the tumor by reducing the temperature of the target tissue to about 20° C. to about 35° C. In some embodiments, the target tissue is reduced to about 20° C. to about 30° C. In some embodiments, the target tissue is reduced to about 20° C. to about 25° C. In some embodiments, the target tissue is reduced to about 25° C. to about 35° C. In some embodiments, the target tissue is reduced to about 21° C. to about 30° C. In some embodiments, the target tissue is reduced to about 22° C. to about 30° C. In some embodiments, the target tissue is reduced to about 23° C. to about 30° C. In some embodiments, the target tissue is reduced to about 24° C. to about 30° C. In some embodiments, the target tissue is reduced to about 25° C. to about 30° C. In some embodiments, the target tissue is reduced to about 30° C. to about 35° C. In some embodiments, the target tissue is reduced to about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., or about 35° C.

In some embodiments, treating the tumor includes cycling between activating the thermoelectric modulator for a period of time and deactivating the thermoelectric modulator for a period of time. In some embodiments, the period of time of activation is minutes, hours, days, or weeks. In some embodiments, the period of time of deactivation is minutes, hours, days, or weeks.

In some embodiments, treating the tumor or other target tissue includes adjusting one or more device parameters to modulate heat transfer from the tumor or other target tissue, thereby increasing or decreasing the temperature of the tumor. In some embodiments, modulating heat transfer decreases the temperature of the tumor or target tissue as compared to a base temperature. In some embodiments, modulating heat transfer increases the temperature of the tumor or target tissue as compared to a base temperature. The device parameters that can be adjusted to modulate the temperature of a target tissue include, but are not limited to, power input to the device (e.g., degree of activation of one or more Peltier modules), the degree of activation of the heat exchange system (e.g., more or less heat transfer), conductance of the probes, the number of probes activated in a multi-probe configuration, and the like.

In some embodiments, the method further includes subjecting the subject to a magnetic resonance imaging (MRI) procedure. As would be recognized by one of ordinary skill in the art based on the present disclosure, one advantage of the systems and devices of the present disclosure is that they can be configured to be MRI-compatible (e.g., reduced metal and/or magnetic components). That is, subjects who may receive therapy with the thermoelectric devices provided herein include subjects with brain tumors; since these subject often require monitoring with an MRI procedure, it is particular advantageous to have a treatment device that is MRI-compatible so that once implanted, it does not have to be removed for imaging. As described further herein, one or more components of the thermoelectric devices of the present disclosure can be configured to be MRI-compatible compatible (e.g., reduced metal and/or magnetic components).

In some embodiments, the subject is receiving or is scheduled to receive another form of treatment for a particular disease or condition. In some embodiments, the subject is receiving or is scheduled to receive chemotherapy. In some embodiments, the chemotherapy includes administration of temozolomide. In some embodiments, the subject is receiving or is scheduled to receive cancer immunotherapy. In some embodiments, the cancer immunotherapy comprises administration of CAR T cells, checkpoint inhibitors, or anti-body-based treatment. Chemotherapy and/or cancer immunotherapy can be administered before, during, or after treatment with the thermoelectric devices provided herein. In some embodiments, the subject is receiving or is scheduled to receive radiotherapy. In some embodiments, focal hypothermia can sensitize tumor cells to radiotherapy. As described further herein, treatment with the thermoelectric devices provided herein have been surprisingly been shown to be compatible with these common cancer treatment modalities, and as such, treatment with the thermoelectric devices of the present disclosure can be used in combination with other treatments in order to provide enhanced therapy.

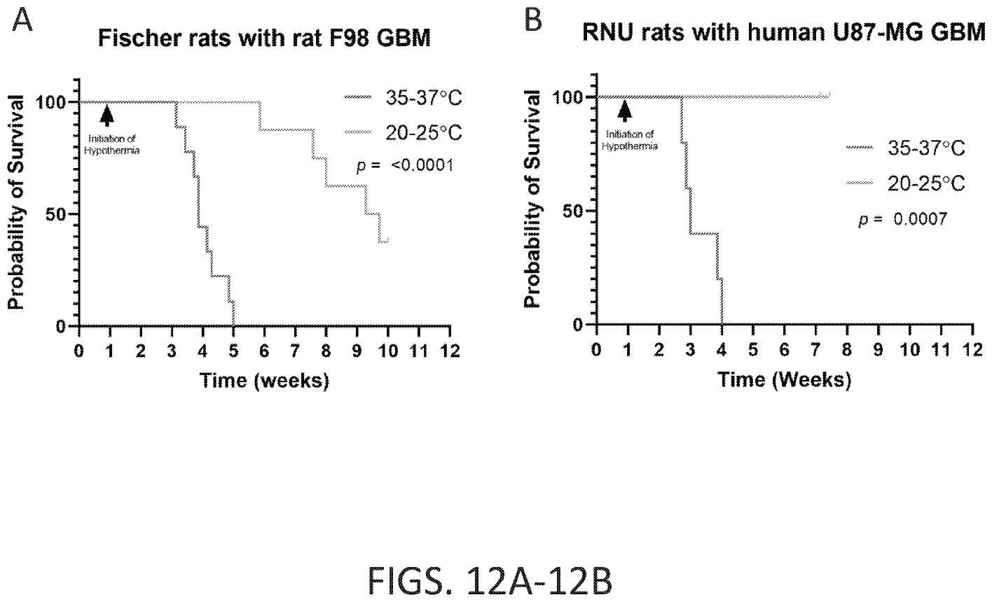

As shown in FIGS. 12A-12B, embodiments of the present disclosure have demonstrated a hypothermia window that doubled rat median survival with an F98 GBM while enabling the rat to eat and move freely and without any gross adverse event due to the hypothermia. By slowing down cell division, the rate of tumor evolution is effectively reduced. Importantly, in patients, this could not only extend survival but also allow for more time to assess adjuvant therapies. As described further herein, this window of safe but growth-halting temperatures is referred to as “cytostatic hypothermia.”

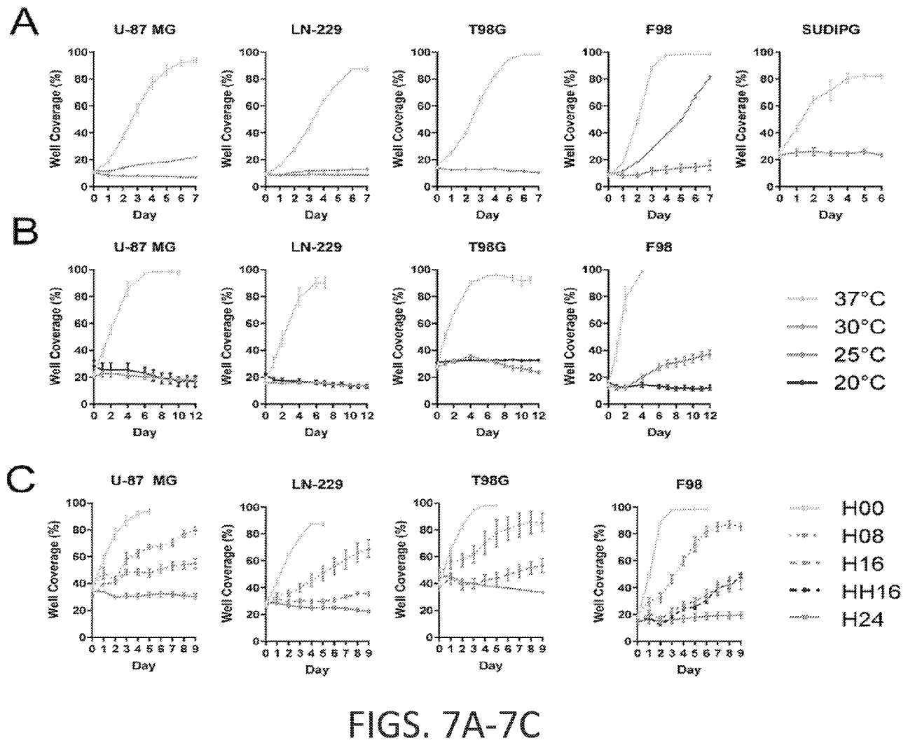

Hypothermia as a therapy is an age-old concept but as a therapeutic for cancer it has only been used at subzero temperatures to obliterate cells. Data provided herein demonstrated cytostatic temperatures across multiple GBM lines ranging from 20-25° C. (FIG. 7). This corroborates some early findings including reduced or halted cell division at temperatures below 28° C. However, in some cases, it was observed that rat F98 tumor cells required a colder temperature to halt division (FIG. 7B). This suggests that cell lines may have different critical pathways that are differentially affected by hypothermia.

Additionally, experiments described herein investigated the effect of intermittent or cycled hypothermia to begin to determine the daily dose of hypothermia required to maintain cell arrest. Results demonstrated that cell arrest for LN-229 may be achieved with 16 h/day, but other cell lines will require 20-22 h/day (FIG. 7C). This is an interesting finding and could have ramifications when translated in vivo. First, for example, this suggests that any in vivo delivery need not run 24 h/day. Furthermore it has been observed that tumors in vivo tend to grow slower than tumors in vitro.

Experiments were also conducted to investigate mechanistic changes and effects of hypothermia. First, these data were consistent with other findings that hypothermia halts cells in the G2-phase of the cell cycle. Ultimately, as the cells are unable to complete division, they undergo apoptosis. In some experiments (e.g., FIG. 7A), a progressive decrease in well coverage was observed, likely due to reduced cell morphology, and possibly apoptosis. Next, ATP levels were assessed to begin to understand metabolic alterations in the cells. It was hypothesized that both production and consumption would diminish but, based on the data, it is possible that consumption diminished more than production. This corroborates other literature in hepatocytes where certain metabolites are differentially affected as either production or consumption are more susceptible. These discrepancies open numerous questions about how the metabolic and transcriptomic profile might change under cytostatic hypothermia. There may be pathways that are more sensitive to hypothermia than others, and this could be elucidated by collecting cell and supernatant samples of different cell lines under progressively colder temperatures.

An acute increase followed by decrease of cytokine production was also observed. The early increased production is likely a stress response coupled with the presence of metabolic resources enabling cytokine production. The subsequent decrease in cytokine production could be due to reduced efficiency of synthetic pathways under hypothermia, or due to reduced metabolic resource usage to power those pathways. The reduction in IL-6 and IL-8 may also be beneficial in cancer as these cytokines are typically associated with aggressiveness and infiltration. However, the absence of anti-inflammatory cytokine secretion under cytostatic hypothermia is promising in certain therapeutic contexts.

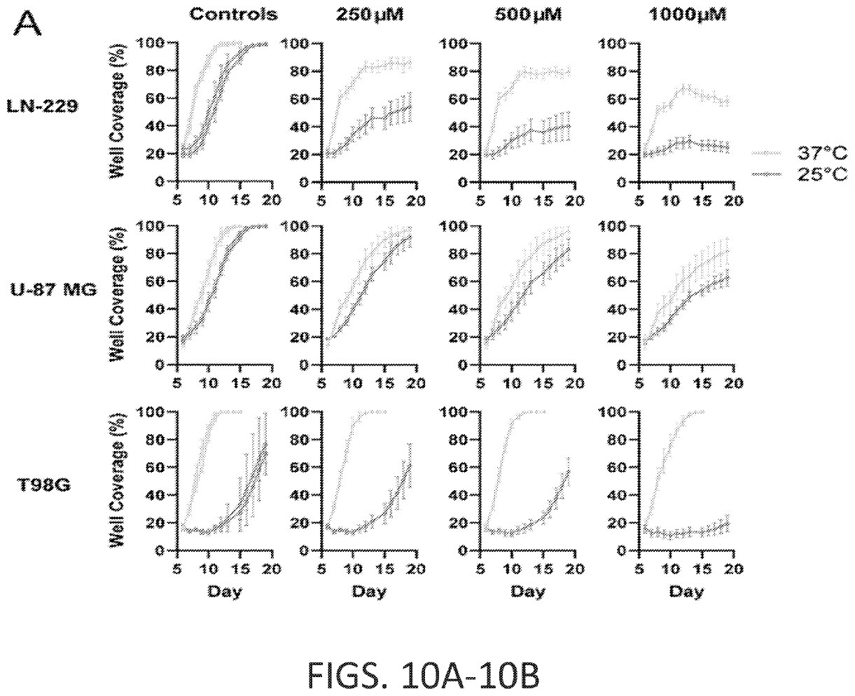

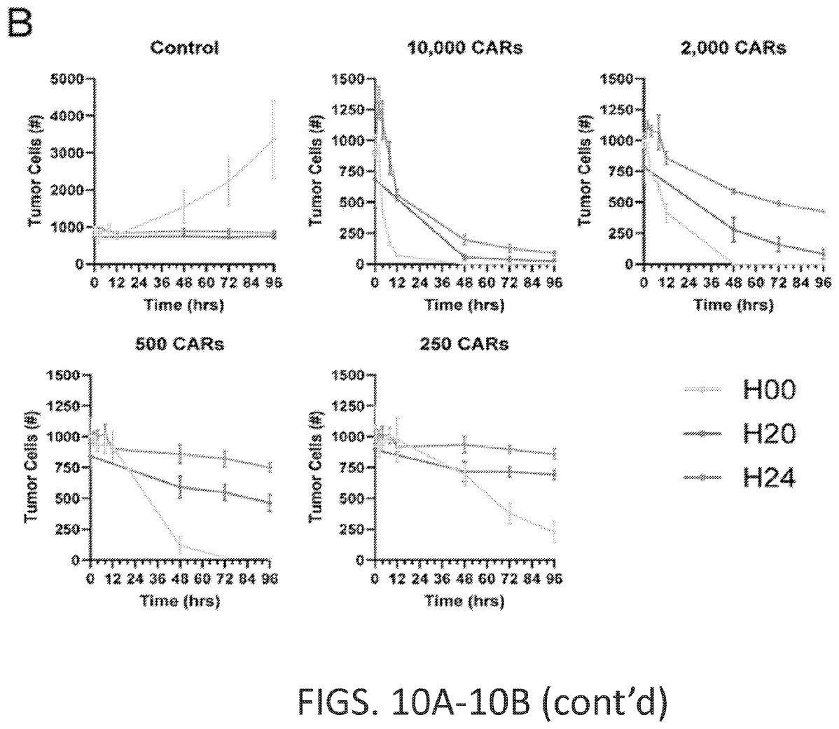

Next, the effect of hypothermia as an adjuvant to a standard-of-care GBM therapy was assessed (i.e. TMZ chemotherapy). TMZ undergoes pH-dependent hydrolysis to form its active ion which methylates guanine residues in the DNA. Attempted correction of this results in double-stranded breaks which halt cell division and ultimately result in apoptosis. Some cell lines, such as T98G, are known to be resistant to TMZ due to the synthesis of an enzyme, MGMT. In the present disclosure, it was observed that, depending on the tumor line, hypothermia might either have no adverse effect or a synergistic effect with TMZ (FIG. 10A). LN-229 were the most synergistically affected, while there was little to no improvement of TMZ effect on U-87. It was observed the most variable results with T98G which may be due to increased sensitivity of the cell line to hypothermia. It is possible that hypothermia either weakened the cells such that they would easily damage during wash or that they are generally less adherent and would come off. Regardless, hypothermia either had little effect or was synergistic with TMZ in the treatment of T98G. These results demonstrate that hypothermia may either have no adverse effect on TMZ or may even be synergistic against some tumors.

As the future of brain tumor therapy seems to lie in modulating the immune response, experiments were conducted to investigate whether immunotherapy could still be used under cytostatic hypothermia. To address this, CAR T cells were used as a model for immunotherapy as they have a direct killing mechanism. It was hypothesized that immune killing efficacy would reduce or completely subside under hypothermic conditions. It was observed that the CAR T cells retained their cytotoxic ability especially at earlier time points. With enough of these effector cells, the tumor was mostly obliterated over 4 days under cytostatic hypothermia (versus 2 days under normothermia). Additionally, by providing only 4 hours of normothermia, the efficacy was improved. These results are interesting as they can be used in at least two approaches. First, an ideal hypothermia duration could be determined to keep tumor growth at check, while providing short windows of normothermia to enable CAR T mediated killing. Second, since CAR T cells seem to function well in the first 24 hours even under hypothermia, multiple doses of CAR T cells could be given while keeping the tumor under constant cytostatic hypothermia. Additionally, data described herein demonstrates that human GBM halt around 25° C. (although rat F98 cells require between 20-25° C.). It should be noted that, until now, all hypothermia data has typically been obtained in the context of intermittent hypothermia or continuous hypothermia for a very short period. However, embodiments of the present disclosure can include cycling hypothermia with normothermia or using adjuvant therapies to reduce the total length of therapeutic hypothermia.

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

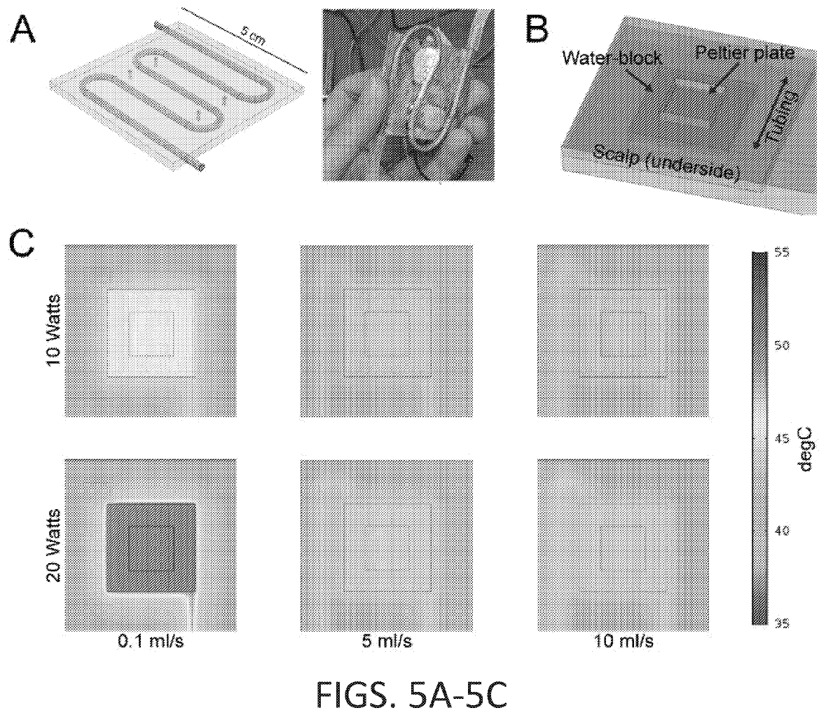

Fully implantable device to deliver focal hypothermia. In exemplary embodiments, the thermoelectric devices and systems of the present disclosure can include three main components: an implantable water-block with internal circulation to transfer heat from a Peltier plate; a multi-probe platform to homogenously apply focal hypothermia in a region of interest; and a multi-Peltier system attached to the platform to enable individual probe control for therapy and research. Combining all the described subcomponents results in a fully implantable thermoelectric device, as shown in FIGS. 4A-4D. The device disclosed herein is made of gold-plated copper and includes a water-block connected to a circulation system, embedded Peltier, and multi-probe array (FIGS. 5A-5B). A diagram of the scale of this device to the skull is depicted in FIG. 4C, but this can be optimized individually for each patient and each therapeutic scenario. While the proposed design is flat for simplicity, it may be modified and designed to incorporate the skull curvature of a patient (FIG. 4D).

Biomedical applications of focal hypothermia typically include an element/probe that is cooled either via circulating liquid or through the thermoelectric effect. In the former, cold liquid is run through a pipe that directly or indirectly contacts the region of interest and heat is transferred from the tissue to the liquid. In the latter, electricity is applied to a Peltier plate to pump heat from the cold side, in contact with tissue, to the hot side. In both these situations, the larger problem involves how to remove the heat from the warmed liquid or the hot side of the Peltier. Current solutions consist of using external ice-baths or secondary external Peltier systems. However, these can have numerous limitations, such as cooling pipes/heat transfer pipes that pass through the skin cause infections; any liquid/ice-bath requires frequent replacement; and power draws from additional Peltier systems would necessitate large batteries.

Embodiments of the present disclosure, however, address these limitations, by providing a fully implantable system to transfer the heat away from the hot side of a Peltier module. In some embodiments, the thermoelectric cooling devices of the present disclosure include a sheet(s) or block of material (e.g., copper, gold-plated copper, graphite, or titanium) with a pipe(s) running through it as a passage for liquid at body temperature (e.g., 37° C.). The metal, in contact with a Peltier module, takes heat and passes it to the liquid. This liquid is circulated through a flexible piping system that is implanted under the skin to distribute the heat throughout other regions of the body. This could either be through contact of the piping system with the skin, or through one or more remote heat sinks/water blocks implanted in the body. The circulation is enabled through an implanted pump that consists either of a traditional motor or, if MRI-compatibility is desired, an induction or electromagnetic motor or a non-magnetic motor such as a piezoelectric motor. This pump is connected to an internal battery which can be charged through wireless electromagnetic induction (or, in simpler form, through a power cable passing through the skin). Its speed can be controlled to increase/decrease the rate of fluid passing through the heat sink, depending on how much heat needs to be removed.

When electricity is applied to a Peltier module, its efficacy is, in part, determined by how well the heat is removed from the hot side. If there is no heat sink, the temperature can reach >70-80° C. before it stops functioning. By reducing and maintaining the hot-side temperature to near body temperature through this system, the Peltier module can become effective.

As provided in FIGS. 5A-5B, embodiments of the present disclosure include configurations in which the water-block exists with variations in dimensions and tortuosity of the internal pipe(s). A simplified model of the water-block on the underside of the scalp (FIG. 5B) was studied with a Finite Element Model (FEM) using coupled multi-physics equations including the Pennes' BioHeat equation (heat transfer) and laminar flow (fluid flow). The FEM was studied at two heat fluxes (10 W and 20 W), both above the required heat flux to cool a 3-cm sphere of brain tissue. Fluid flow was tested at 3 rates: 0, 3, and 30 ml/s. Results demonstrate that, without fluid flow, local temperature at the scalp can reach temperatures of 55-65° C. with 10 and 20 W, respectively. The addition of fluid flow/circulation through the system, can significantly bring down the temperature, with 30 ml/s flow reducing the scalp temperature to 38 and 39.5° C. at 10 W and 20 W, respectively (FIG. 5C). As shown, even at 20 W, 10 ml/s induces a max temperature of around 40° C. (FIG. 5C).

Additionally, Lab testing of a prototype water-block (FIG. 5A) at room temperature demonstrated that a Peltier plate can achieve at least −15° C. on the cold side when water of 37° C. is passing over the hot-side. These results suggest that an implantable water-block cooling system with a non-local pump to enable fluid circulation can effectively remove heat from a Peltier plate and enable local cooling.

### Example 2

Removing heat from a region of tissue (e.g., brain), through a probe or surface, can be limited by the amount of surface area in contact with the tissue. With reduced surface area, the temperature within the probe or surface must be significantly colder (and more energy required to maintain it) so that the tissue reaches the desired temperature. However, this forms sharp thermal gradients, wherein the tissue adjacent to the probe is much colder so that tissue further away can also reach a desired temperature.

To address this problem of thermal gradients and excessively cold temperatures around probes, a multi-probe heat distribution platform was developed (see, e.g., FIG. 4 and FIG. 6). Embodiments of this platform can include a metal/sheet/vapor chamber through which needles or heat pipes can emerge in an array. The array can be any montage necessitated for a given application/problem. The array could also consist of insulated portions to limit cooling in undesired regions and increase cooling efficiency in desired regions.

As provided in FIG. 6, an FEM using the Pennes' bioheat equation was developed with a goal to cool the outer surface of a 3-cm sphere of highly blood perfused tissue (residual tumor in brain) to at least 25° C. A heat flux of 8 W across the platform supporting either a 1×1 or 5×5 probe array was set (chosen from preliminary studies). Using a single probe (1×1) necessitated the probe to be much larger in diameter, 1.5 cm, and brought tissue adjacent to the probe to below 0° C. (FIG. 6A). Instead, using a 5×5 probe array, each 0.5 mm in diameter, the coldest recorded temperature was 18° C. anywhere within the sphere, while the highest was 25° C. The total volume of tissue displaced was also significantly lower between the single probe and 5×5 probe array: 7000 mm³ vs. 200 mm³. Thus, using a multi-probe array instead of a single probe not only resulted in a more homogenous temperature distribution, but also had a smaller footprint.

### Example 3

In some cases, different regions of tissue may need to be cooled at different intensities. This could be due to contact with tissue (or nearby tissue) that is critical or the need to be more efficient with power usage.

Therefore, instead of having one large cooling Peltier plate in contact with the multi-probe platform, a multi-Peltier module device was developed wherein each probe is controlled by an individual Peltier (FIGS. 6C-6D). This embodiment enabled finer control over the extent of cooling. Each Peltier can be individually controlled. To limit the amount of heat leaking between probes and Peltier plates, insulation material can be incorporated to isolate each.

This application is not limited to therapy in patients. It can also be used as a neuroscientific tool to study the effect of cooling and heating on brain function in animals; analogous to a multi-electrode array to read/control individual neurons.

### Example 4

In vitro tumor growth rate is hampered by varying depths and durations of hypothermia. It was hypothesized that different depths of hypothermia may affect cell growth rates differently. Thus, three human GBM cell lines and one rat GBM line were cultured at 20, 25, and 37° C., while also culturing some at 30° C. (FIGS. 7A-7B). Growth was assessed daily through a custom imaging and analysis method. All cell lines grew to 95-100% confluence within 3-5 days. Reducing the temperature to 30° C. reduced the growth rate of all cell lines tested. The three human GBM lines, and a human DIPG line, all showed no growth at 25° C. (FIG. 7A). Interestingly, this was not the case with the F98 (rat GBM) cell line which, while significantly reduced, still demonstrated growth. Further reducing the temperature to 20° C. halted F98 growth (FIG. 7B).

To assess the effects of hypothermia applied for 24 hours per day, the plates were cycled between 37° C. and 25° C. incubators for varying durations. This demonstrated that even 16 hours of cooling (H16) could significantly reduce growth rate (FIG. 7C). Application of hypothermia tended to alter the morphology of the cells wherein they became more spherical within 24 hours.

### Example 5

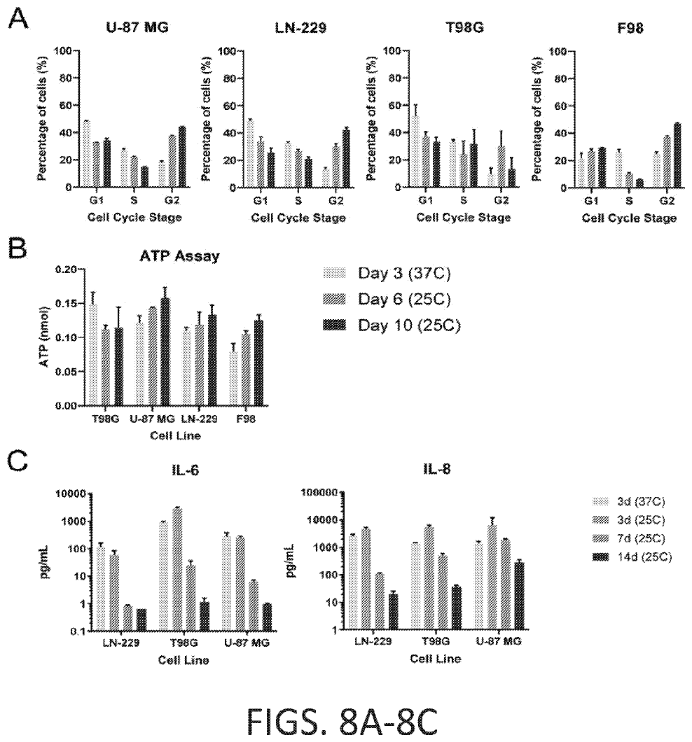

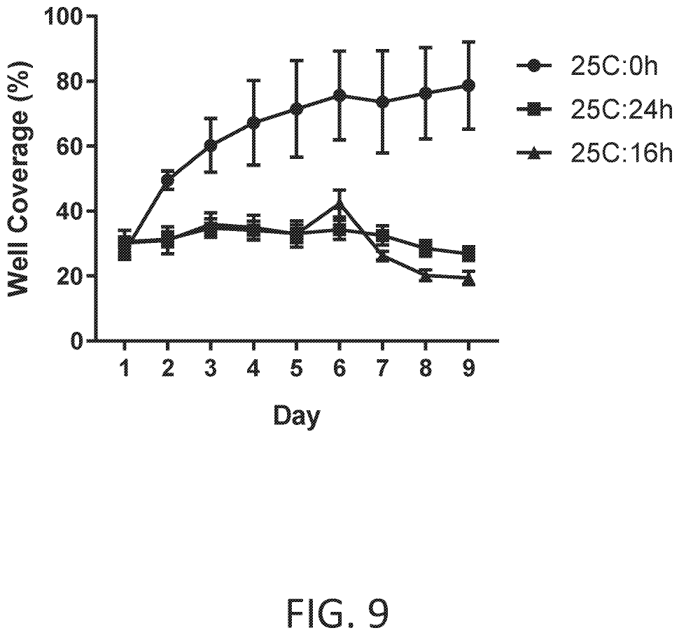

Hypothermia halts cell cycle, and affects ATP stock and cytokine production. To begin to understand the mechanism and effects of halted cell division, cell cycle, ATP levels, and cytokine production were assessed under hypothermia. A larger proportion of cells grown for 3 days under 37° C. were found to be in the G1 phase of the cell cycle (FIG. 8A). However, with 6 or 10 days of cytostatic hypothermia, the proportion shifted towards the G2-phase (FIG. 8A). Interestingly, the one cell line that does not halt division, F98, did not show a significantly higher G1 phase at 37° C. compared to the other groups. However, it too had more cells in the G2-phase. This suggests that hypothermia prevents cell division from completing.

Next, ATP levels were studied to begin exploring metabolic alterations. It was hypothesized that, under cytostatic hypothermia, ATP production and consumption would cease and thus intracellular ATP levels would remain constant. Interestingly, an increase in ATP levels in 3 out 4 cell lines under hypothermia was observed (FIG. 8B).

Additionally, to assess the effect of hypothermia on cytokine production, IL-6, IL-8, CX3CL1, IL-10, IL-4, and IFNγ were measured. It was hypothesized an ‘acute’ increase in anti-inflammatory cytokines followed by a decrease over time. Interestingly, the latter 3 cytokines were not found in the conditioned media regardless of the temperature (e.g., suggesting anti-inflammatory cytokines were not suddenly increasing). Additionally, inflammatory cytokines IL-6 and IL-8 increased at 3 days of hypothermia but drastically reduced over 7 and 14 days across all 3 cell lines assessed (FIG. 2C).

### Example 6

Hypothermia treatment with chemotherapy and CAR T immunotherapy treatment. Current standard of care for GBM includes chemotherapy with Temozolomide (TMZ). Additionally, the future of many cancer therapies includes some form of modulating the immune response; an immunotherapy. To assess whether hypothermia would hamper or synergize with Temozolomide, human GBM cells were treated with different TMZ doses either at 37° C. or 25° C. for 3 days followed by washing the cells and growing at 37° C. Interestingly, the responses of the cell lines were different. The addition of TMZ to LN-229 cells under hypothermia reduced cell division more significantly than under 37° C. and compared to cells that did not receive TMZ (FIG. 10A). U-87MG was also mildly affected by TMZ under hypothermia, but whether the growth rate difference was due to the synergy or due to hypothermia alone cannot be confirmed as hypothermia alone also delayed growth (FIG. 10A). The most variation to response was observed with T98G cells. T98G cells under hypothermia tended to be affected by washing wherein the cells would be damaged and wash off (while leaving cell membrane debris on the plate).

Next, the effect of hypothermia on CAR T immunotherapy against EGFR+CT2A mouse GBM was explored. It was hypothesized that immune cell function would be significantly hampered but may retain some killing activity due to accumulated ATP. It was also hypothesized that killing efficacy would increase if the temperature were cycled between 25 and 37° C. Synthesized CAR T cells were functional and specifically killed EGFR+CT2A cells (and not EGFR−). Absence of the CAR on T cells resulted in no killing of any CT2A cell. Without CAR T cells, CT2A cell growth was inhibited by 25° C. hypothermia both when the hypothermia was applied for 24 h/day and 20 h/day (FIG. 10B). With the addition of 10,000 CART cells (10:1 ratio of T cell:tumor), the CART cells eradicated tumor within 36-48 hours at 37° C. Interestingly, the application of 20 h/day hypothermia eradicated tumor within 72-96 hrs (FIG. 10B), and 24 h/day hypothermia almost eradicated tumor within 96 hrs (FIG. 10B). Reducing the number of CAR T cells reduced the killing efficacy regardless of temperature. These results suggest that while hypothermia can reduce the efficacy of tumor killing, fresh immune cells can still engage in killing early on. Additionally, temperatures can by cycled to increasing killing efficiency.

### Example 7

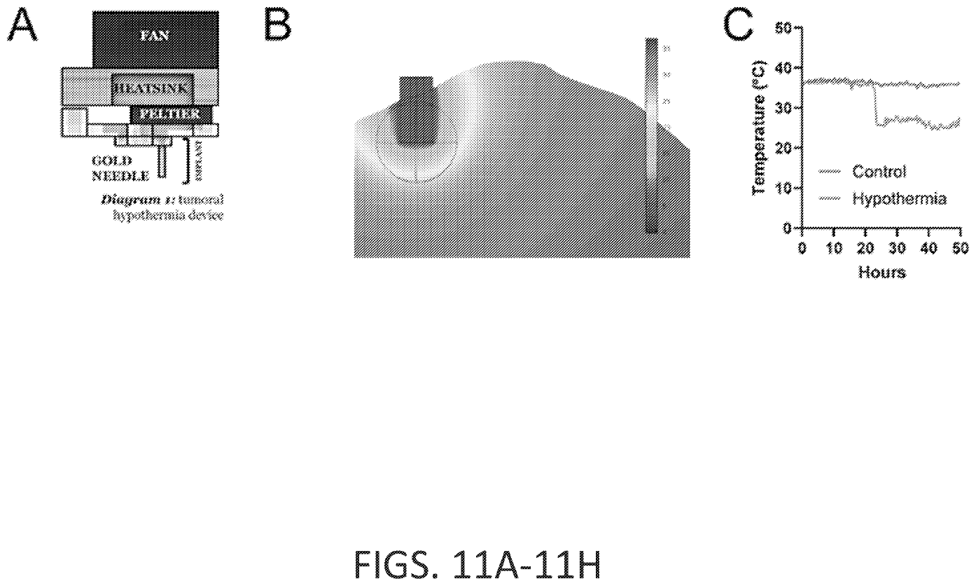

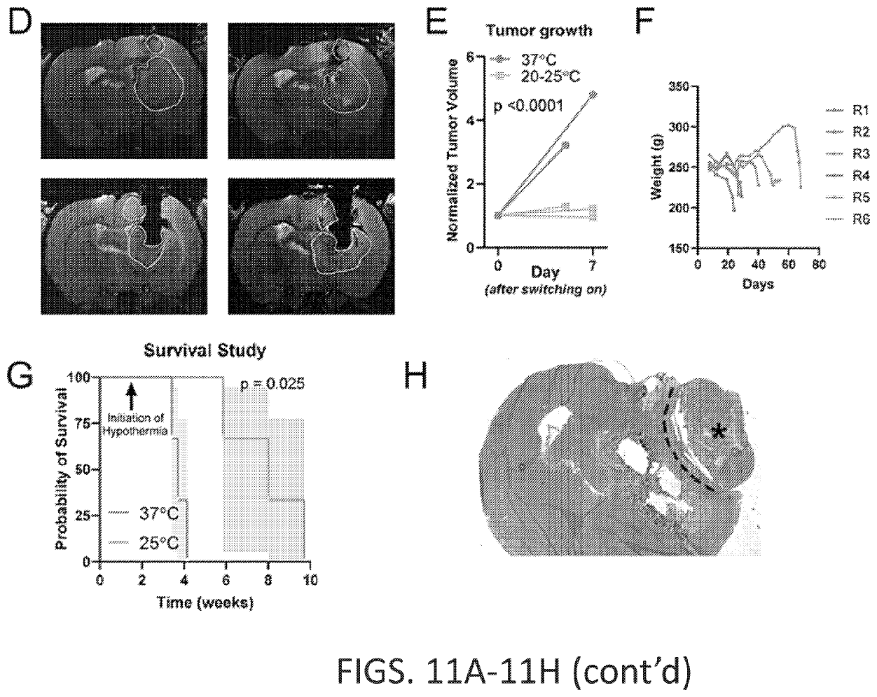

Focal hypothermia in vivo prolongs survival of GBM bearing rats. Since hypothermia of 20-25° C. can reduce cell division in vitro, and this temperature range may be safe in vivo, a method was developed to deliver hypothermia in vivo and assess its effect on tumor growth and rat survival. To deliver hypothermia, a Peltier based device was designed and developed that was comprised of an implantable portion (including an intratumoral gold needle, a thermistor 1-mm from probe, and polycarbonate base) and a removable portion (including the Peltier, aluminum heatsink and fan) (FIG. 11A). This device was then powered through an external power supply and temperature was recorded with an Arduino and computer. A custom cage and cabling set up was developed to ensure free movement of the rats.

A finite element model was set up to assess the extent of cooling in vivo (FIG. 11B). This model demonstrated that the extent of cooling is primarily influenced by the rate of blood perfusion. In a rat brain, by cooling a probe to 0° C., a 2-mm diameter tumor can reach <25° C. on its periphery. The temperature returns to 37° C. about 5 mm away from the probe.

After inoculation with the aggressive F98 tumor cells, tumor take was confirmed in all rats via MRI at week, and the device was implanted on the subsequent day. Once switched on, the device was able to reduce local temperature to at least 25° C., 1 mm away from the intratumoral probe (FIG. 11C). In one set of studies using an MRI-compatible thermistor, tumor growth was assessed at 1 week via MRI (FIGS. 11D-11E). This demonstrated a large and significant difference in tumor volume for tumors under hypothermia versus tumors under body temperature.

In subsequent survival studies, rats receiving hypothermia did not exhibit any obvious signs of distress and ate their normal diet. This translated to longer weight maintenance and gain compared to their control counterparts (FIG. 11F). During the study, regular device maintenance was required and there were periods of device failure in the treatment rats which were corrected. Application of hypothermia significantly doubled the median survival of rats with the F98 GBM tumor (FIG. 11G). The rats with the device switched off (control) reached euthanasia criteria within 1 week (3-4 weeks) of each other. The first treatment rat with hypothermia reach the criteria at 6 weeks. Upon histological examination, an extradural tumor was observed (FIG. 11H). This misplacement likely occurred during inoculation and suggests that the cause of early death was a tumor that was not receiving cooling. The other two rats had periods of device failure but still managed to survive at least twice as long as the control group. This study demonstrates that focal delivery of cytostatic hypothermia may prolong symptom free life.

### 5. Materials and Methods

Cell Culture. All cell lines were purchased from either ATCC or the CCF at Duke. To simplify culturing and passaging, all cells were progressively adapted to a unified medium: Dulbecco's MEM (with glucose and Na pyruvate)+NEAA+10% FBS. This might affect growth rates for individual cells and thus future studies can characterize the effects of other media with cytostatic hypothermia. Cells were given at least one passage to recover after thawing and then used within the next 5-10 passages for all experiments. Cells were plated at a density of 5000 cells/well in falcon clear-bottom 96-well plates or 10,000 cells/well in a 24-well plate and left to adhere overnight at 37° C. before any experiment. In certain experiments, cells were left for longer at 37° C. prior to changing incubator temperature. A HeraCell CO2 incubator was used for all experiments and set at either 37, 30, 25, or 20° C.

Imaging and analysis. Imaging was performed with a Leica DMi8 live cell scope with a built-in incubator and CO2 regulator. Leica software was calibrated to the imaging well plate to enable tile scanning. Images were taken at 5× with a 4×4 field in 24-well plates and 2×2 field in 96-well plates. Images were then analyzed through a custom automated imageJ script to quantify the area coverage of a well plate. For immunotherapy experiments, images were taken at 10× with a GFP laser, and the imageJ script was modified to count the number of cells. All analyzed images were then processed through a custom Python script to organize the data for analysis.

Chemo/immunotherapy. Chemotherapy: temozolomide (sigma Aldrich) was dissolved in DMSO and frozen in aliquots and used within 2 months at varying concentrations. In these experiments, tumor was grown in the well plate overnight followed by treatment of 37 deg plate with TMZ for 3 days. The second plate was moved to 25° C. for 5 days. After 5 days, TMZ was added for 3 days. After TMZ treatment, the media was replaced and the plates were moved back to 37° C. with daily imaging.

CAR T cells were made by harvesting splenocytes and isolating T cells. HEK cells were transfected with a plasmid to produce lentivirus with EGFR+. This media was then used to transduce the T cells to express the CAR. CAR was confirmed via flow cytometry and the cells were frozen in liquid nitrogen. For in vitro experiments, all experiments were on days 5 or 6 after transduction.

Device manufacturing. Device design and manufacturing was done in collaboration with the Pratt Machine Shop at Duke University. Custom designs were made and transferred to 3D CAD drawings. Raw polycarbonate, copper, and aluminum material was purchased from McMaster and parts were machined using a CNC machine. Other components include Peltier plates from TeTech, fans from Sunon (purchased through supplier: Digikey), thermistors from Amphenol Advanced Sensors, 24K Gold from Hauser & Miller, thermal paste (Kryonaut) from Thermal Grizzly, screws from McMaster, and connectors from JST Sales America (purchased through supplier: Digikey).

The implant consists of a gold needle, copper base, and thermistor that warps around brass screw posts. This and other components were sterilized through UV for 30 minutes followed by EtOH overnight.

Animals. All animal procedures were approved by the Duke IACUC. Fischer (CDF) rats and RNU rats were purchased from Charles River at 6-9 weeks of age. All procedures began at 10 weeks of age. The animals were induced with 5% isoflurane and maintained at 2%. A central incision was made on the scalp and the skin mildly retracted. A 0.6 mm conical burr was used to drill at −0.5 AP and +3 ML to a depth of 0.8-0.9 mm. A Hamilton syringe loaded with at least 5 uL of F98 or U87MG cells was centered to drill site and then the tip was cleaned of any droplets. The syringe was lowered to a depth of 1.5 mm from the outer table. Infusion was begun with a pump at 0.5 uL/min for 10 min. Upon 1 minute after completion, the syringe was slowly retracted and the rat scalp sutured. The rat was then placed in the custom cage.

One week after, an MRI was taken to confirm tumor-take. The subsequent day, the rats were again induced under anesthesia and the scalp exposed. This time, extra effort was put into retraction, scraping off the peritoneum, slightly separating the termporalis muscles, and hemostasis. Once the cranium was dry and absent of blood, additional burr holes were made using conical drill bits. This included one burr hole with 0.6 mm tip for thermistor, −1.5 mm from tumor inoculation. Following this, 1.0 mm conical burr was used −6 mm from thermistor for titanium screw (TS) 1, −4 mm from TS1 for TS2, −10 mm from thermistor for TS3, and +6 mm from tumor inoculation for TS4. The original tumor inoculation burr hole was expanded to 1.4 mm. Screws (modified to be 1 mm in length) were then twisted into their holes at a depth of 0.6 mm. Next, the sterile implant was gently inserted and held down while dental cement was added to the sides. As quickly as possible to hold the implant, a UV light was shone. Following this, layers of dental cement were added around the screws and implant and skull to secure the implant to the skull. Upon completion, stitches were used to gently approximate the skin (including around the arms of the implant) while keeping the surface of the implant exposed.

The rat was monitored while waking up and for 2 hours after to ensure full recovery. Two days after recovery, the rat was put under anesthesia to screw on the remaining device and connect the cable to the slip-ring on the cage. For studies where MRI was possible, an additional MRI was taken 5 days after implant (with the device screwed off). After this, the device was switched on, and temperature was monitored through an Arduino connected to a computer.

The rat temperatures were intermittently monitored throughout the day through a local network. This enabled rapid responses to any sudden changes in temperature, usually due to some transient failure of the device which was rectified in future iterations. One gradual source of inefficiency was the buildup of fur on the intake of the heat sink and inside the fan. This needed to be intermittently removed with tweezers.

Rat weight was measured every 3-7 days and every day when weight started falling. The procedure involved transiently disconnecting the rat from the tether, moved to an empty cage, and subtracting the weight of the cable/components/and cage. the rat lost 15%

MRI. For MRI, the cooling portion of the device was detached under anesthesia. The rats were imaged in a Bruker 7T MRI machine. T1 and T2 weighted images were taken. Contrast was also added for the T1 images through a tail vein catheter inserted under anesthesia right before the MRI.

Euthanasia/perfusion/histology. Euthanasia criteria included: 15% weight loss from initial weight (post device implant), and signs of porphyrin staining and distress. When a rat reached these criteria, they were put under anesthesia. A thoracotomy was performed and the rats were then transcardially perfused with saline (250 mL) followed by fresh 4% formalin (250 mL). The animals were decapitated, and the skull and implant was carefully removed. The brain was left for 24 hours overnight in formalin. The following day they were transferred to 30% sucrose and left until the brain sunk. For histology, the brain was snap frozen in liquid nitrogen, cryosectioned at 12 um slices, stored on slides and placed in −20° C. The slides were stained with H&E and imaged under the microscope.

Computational modelling. Modelling was performed on COMSOL v5.2.

Statistical analysis. All statistical analysis was performed on Graphpad prism v8.2.

What is claimed is:

1. A thermoelectric cooling device for reducing temperature of a target tissue in a subject, the device comprising:
a thermoelectric modulator;
a heat exchange system functionally coupled to the thermoelectric modulator; and
at least one thermally conductive probe functionally coupled to the thermoelectric modulator;
wherein activation of the thermoelectric modulator transfers heat from a target tissue to the heat exchange system, thereby reducing the temperature of the target tissue.

2. The device according to claim 1, wherein the thermoelectric modulator comprises a Peltier module.

3. The device according to claim 2, wherein the Peltier module comprises a Peltier cell coupled to the at least one thermally conductive probe.

4. The device according to claim 1, wherein the thermoelectric modulator comprises a plurality of Peltier modules, wherein each Peltier module comprises a Peltier cell coupled to the at least one thermally conductive probe.

5. The device according to any of claims 2 to 4, wherein the Peltier module comprises a thermally conductive base plate positioned between the Peltier cell and the at least one thermally conductive probe.

6. The device according to any of claims 1 to 5, wherein activating the thermoelectric modulator comprises applying electrical power to the thermoelectric modulator to cause heat to be transferred from the target tissue to the heat exchange system.

7. The device according to any of claims 1 to 6, wherein the heat exchange system comprises a fan to facilitate the transfer of heat from the target tissue.

8. The device according to any of claims 1 to 6, wherein the heat exchange system comprises a fluid block to facilitate the transfer of heat from the target tissue.

9. The device according to claim 8, wherein the fluid block comprises a piping system and a fluid, wherein the piping system is coupled to the thermoelectric modulator and wherein the fluid flows through the piping system, thereby facilitating the transfer of heat from the target tissue.

10. The device according to claim 9, wherein the fluid comprises a biocompatible coolant.

11. The device according to any of claims 1 to 10, wherein the thermoelectric modulator comprises a power source.

12. The device according to any of claims 1 to 11, wherein the heat exchange system comprises a magnetic motor or a non-magnetic motor.

13. The device according to any of claims 1 to 12, wherein the at least one thermally conductive probe is in direct contact with the target tissue.

14. The device according to any of claims 1 to 13, wherein the at least one thermally conductive probe is comprised of a biocompatible material.

15. The device according to any of claims 1 to 14, wherein the device further comprises a target tissue temperature monitoring device configured to be in direct contact with the target tissue.

16. The device according to any of claims 1 to 15, wherein the device further comprises a control system, wherein the control system is configured to modulate the temperature of the target tissue by adjusting or more device parameters to increase or decrease heat transfer from the target tissue.

17. The device according to any of claims 1 to 16, wherein the temperature of the target tissue is reduced to about 20° C. to about 35° C.

18. The device according to any of claims 1 to 17, wherein the target tissue comprises a tumor.

19. The device according to any of claims 1 to 17, wherein the target tissue comprises a glioblastoma.

20. A method of treating a target tissue in a subject, the method comprising activating the device of any of claims 1 to 19, wherein activation of the device transfers heat from the target tissue to the heat exchange system, thereby reducing the temperature of the target tissue.

21. A method of treating a tumor in a subject, the method comprising:
implanting a thermoelectric cooling device in a subject, wherein the device comprises a thermoelectric modulator, a heat exchange system functionally coupled to the thermoelectric modulator, and at least one thermally conductive probe functionally coupled to the thermoelectric modulator and in direct contact with the tumor; and
activating the thermoelectric modulator, wherein activating the thermoelectric modulator transfers heat from the tumor to the heat exchange system, thereby inducing cytostatic hypothermia in the tumor.

22. The method according to claim 21, wherein inducing cytostatic hypothermia in the tumor comprises reducing the temperature of the tumor to about 20° C. to about 35° C.

23. The method according to claim 21 or claim 22, wherein treating the tumor comprises cycling between activating the thermoelectric modulator for a period of time and deactivating the thermoelectric modulator for a period of time.

24. The method according to any of claims 21 to 23, wherein treating the tumor comprises adjusting one or more device parameters to modulate heat transfer from the tumor, thereby increasing or decreasing the temperature of the tumor.

25. The method according to any of claims 21 to 24, wherein the method further comprises subjecting the subject to a magnetic resonance imaging (MRI) procedure.

26. The method according to any of claims 21 to 25, wherein the subject is receiving chemotherapy.

27. The method according to claim 26, wherein the chemotherapy comprises administration of temozolomide.

28. The method according to any of claims 21 to 25, wherein the subject is receiving cancer immunotherapy.

29. The method according to claim 28, wherein the cancer immunotherapy comprises administration of CAR T cells, checkpoint inhibitors, or anti-body-based treatment.
